(19) United States
(12) Patent Application Publication
(10) Pub. No.: US 2008/0207754 A1
(43) Pub. Date: Aug. 28, 2008

(54) Method and apparatus for palatable prevention of acid reflux

(76) Inventor: William A. Zicker, Forest Hill, MD (US)

Correspondence Address: Ober, Kaler, Grimes & Shriver;Attorneys at Law, 120 East Baltimore Street, Baltimore, MD, 21202-1643, US

(21) Appl. No.: 12/072,909

(22) Filed: Feb. 28, 2008

Related U.S. Application Data
(60) Provisional application No. 60/903,949, filed on Feb. 28, 2007.

(51) Int. Cl. A61K 31/19; A61P 1/04; A47G 19/22; B65D 1/36

(52) U.S. Cl. 514/557; 220/705; 220/500

(57) ABSTRACT

A method and applicators for preventing or reducing acid reflux (or “GERD”) by delivering a proper dose of vinegar directly to the back of the throat without exposing the taste buds, and immediately followed by a shot of water or other pleasant tasting chaser to wash it down. One applicator generally comprises a joined pair of sealed flexible pouches each sealed by a seal that may be burst open on the application of pressure to the pouches in order to dispense the product therein. The first pouch is filled with a dose of undiluted apple cider vinegar (approximately 5 to 10 cc) and the second pouch is filled with a liquid chaser, usually distilled water. Another applicator generally comprises a drinking vessel defined by two side-by-side receptacles, and pair of fixedly attached straws each entering a corresponding receptacle. The straw entering the chaser is oriented at a fixed angle greater than the straw entering the vinegar so that vinegar pours first followed by the chaser. In use, the vessel is lifted to the mouth with both straws disposed at the back of the throat, and the vessel is tilted so that the vinegar receptacle pours first, followed immediately by the chaser. This results in the vinegar pouring directly down the throat without exposing the taste buds, and being immediately followed by the chaser to wash it down.

### CROSS-REFERENCE TO RELATED APPLICATIONS

The present application derives priority from U.S. Provisional Patent Application No. 60/903,949 for “METHOD AND APPARATUS FOR PALATABLE PREVENTION OF ACID REFLUX”; Filed: Feb. 28, 2007; Inventor: William A. Zicker.

### FIELD OF THE INVENTION

The present invention relates to a home remedy for preventing or reducing acid indigestion and especially acid reflux or Gastroesophageal Reflux Disease (“GERD”), a common condition in which the liquid content of the stomach backs up into the esophagus.

### BACKGROUND OF THE INVENTION

Approximately 5 to 7 percent of the population suffers from chronic acid reflux or Gastroesophageal Reflux Disease (“GERD”) which is marked, in addition to heartburn, by symptoms including coughing, hoarseness, voice change, chronic ear ache, chest pain, nausea and sinusitis. Under normal conditions stomach acid is prevented from entering the esophagus by a muscular valve called the lower esophageal sphincter. If that valve, for whatever reason, fails to operate properly, stomach acid may flow up the esophagus causing damage and the identified symptoms. There is no single cause for the disorder of the esophageal sphincter but contributing factors include smoking, obesity, pregnancy, the use of certain medications and consumption of aggravating foods (which vary on an individual basis).

Patients diagnosed with the disorder are often prescribed proton-pump inhibiting drugs in addition to over the counter antacids to relieve symptoms and control the disorder. Other treatments involve weight loss, restrictive diets and various body positioning recommendations. These efforts, particularly prescription drugs, can be expensive and vary in effectiveness from patient to patient. Extensive anecdotal evidence and patient testimonials indicate that apple cider vinegar provides temporary relief from the symptoms of GERD. Apple cider vinegar contains acetic acid in concentrations ranges typically from 4 to 8 percent by volume as well as smaller amounts of tartaric acid, citric acid, and other acids. The acidic nature of the cider vinegar resulting from the fermentation process produces an astringent effect that tends to shrink or constrict the muscles of the esophageal sphincter on contact causing it to close fully thereby preventing acid indigestion and reflux. A suitable regimen includes two tablespoons in water, three times a day or as needed although the dosage may vary significantly from patient to patient. A bottle of apple cider vinegar cost between $3.00 and $5.00. Patients have reported increased benefit through lower dilution or through use of undiluted vinegar.

A distinct advantage of this remedy is that it is extremely inexpensive, costing only a few cents each day, yet widely effective. However, the ingestion of vinegar is difficult for many, if not most, people as the liquid has a notoriously unpalatable taste and smell. This has proven a disadvantage as the difficulty of drinking even a small quantity has prevented many from taking advantage of the health benefits. In fact, the ingestion of vinegar most often prompts the pharyngeal or gag reflex due to the astringent liquid contacting the soft palate of the mouth. The reflex contracts the back of the throat in order to prevent something from entering except as part of normal swallowing to prevent choking, but has the effect of preventing ingestion of vinegar even when intended by the individual. Some people dilute the vinegar with water to make it more palatable although this is only marginally effective at increasing palatability and tends to reduce the effectiveness of the overall treatment. Some nutraceutical manufacturers now offer apple cider vinegar tablets in an effort to resolve the palatability issue. While these tablets avoid the bad taste, they have been found to cause esophageal injury in some cases. See, Hill, L., et al (2005). “Esophageal Injury by Apple Cider Vinegar Tablets and Subsequent Evaluation of Products”; Journal of the American Dietetic Association, Volume 105 (Issue 7): 1141-1144.

It would be far more advantageous to provide a way of getting the proper dose of undiluted liquid vinegar into the throat without exposing the taste buds, soft palate and olfactory sense so as to avoid the gag reflex, unpleasant taste or burning. The present invention is a method and apparatus for accomplishing exactly that.

### SUMMARY OF THE INVENTION

It is therefore an object of the present invention to provide a method and apparatus for preventing or reducing heartburn or Gastroesophageal Reflux Disease by delivering a proper dose of pungent liquid directly to the back of the throat without exposing the taste buds or soft palate.

It is another object to provide a two-shot applicator for delivering a pre-measured or refillable dose of pungent liquid to the back of the throat immediately followed by a shot of water or other chaser to cleanse the palate and prevent the gag reflex.

It is another object to provide the ability to self-administer a measured unit dose of the pungent liquid and chaser.

In accordance with the foregoing objects, the present invention is a method and applicator embodiments for preventing or reducing acid reflux or GERD by delivering a proper dose of pungent (unpalatable) liquid directly to the back of the throat without exposing the taste buds, soft palate and olfactory sense, immediately followed by a shot of water or other pleasant tasting chaser to wash it down.

The applicator generally comprises a joined pair of sealed flexible pouches similar in nature to those used in food product packaging, particularly in regard to single serving condiment packaging. The paired pouches are joined together at their periphery, or otherwise. Each pouch is fitted with an aperture along one edge which is sealed by a seal that may be burst open on the application of pressure to the pouches in order to dispense the product therein. The first pouch is filled with a pre-measured dose of pungent liquid such as undiluted apple cider vinegar (approximately 3 ml) and the second pouch is filled with pre-measured dose of liquid chaser, such as distilled water or other palatable liquid (approximately 6 ml).

In use, the pouches are folded about their joined edge so as to align the pouches and apertures located thereon. The folded pouches are inserted into the mouth with the aperture end disposed at the back of the throat and squeezed. The applied pressure bursts the seals and dispenses the vinegar and chaser down the throat without exposing the taste buds. The vinegar and chaser may be mixed as they are dispensed or a mechanism may be employed to delay the discharge of the chaser to allow the vinegar dose to be taken in undiluted form. Delay of the chaser may be accomplished by varying the pressure required to burst each seal or by routing the chaser through a discharge conduit increasing the distance the liquid must travel before being discharged and thereby increasing the time to discharge.

In an alternate embodiment, the applicator generally comprises a drinking vessel defined by two side-by-side receptacles, including a first receptacle and a second receptacle. A pair of fixedly attached straws each enter a corresponding receptacle, the straws being fixedly attached to the inner walls of the vessel, opening at the bottom of the receptacles and extending side-by-side to adjacent distal tips at least one inch (and preferably about two inches) remote from the vessel. One of the straws (entering the second receptacle) is oriented at a fixed angle greater than the other of straw (entering the first receptacle) so that contents of the first receptacle pours first immediately followed by the contents of the second receptacle. The first receptacle is filled with a measured dose of pungent liquid, preferably approximately 3 ml of undiluted apple cider vinegar, and the second receptacle is filled with a measured dose of liquid chaser, preferably approximately 6 ml of water.

In use, the vessel is lifted to the mouth with both straws disposed at the back of the throat, and the vessel is tilted so that the vinegar receptacle pours first, followed immediately by the chaser. This results in the vinegar pouring directly down the throat without exposing the taste buds, and being immediately followed by the chaser to wash it down.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a front view of a dispenser 2 according to one preferred embodiment of the invention.

FIG. 2 is a front view of a dispenser 12 according to another preferred embodiment of the invention.

FIG. 3 is an elevation view of the dispenser 22 according to an alternate embodiment of the invention.

FIG. 4 is a diagram of the human mouth and throat illustrating the method of use of the present invention.

FIG. 5 is a perspective view of the applicator 32 according to another alternate embodiment of the invention.

Corresponding reference characters indicate corresponding parts throughout the several views. Although the drawings represent embodiments of various features and components according to the present invention, the drawings are not necessarily to scale and certain features may be exaggerated in order to better illustrate and explain the present invention. The exemplification set out herein illustrates embodiments of the invention, and such exemplifications are not to be construed as limiting the scope of the invention in any manner.

### DETAILED DESCRIPTION OF THE PREFERRED EMBODIMENTS

The present invention is a method and applicators for preventing or reducing acid reflux or GERD by delivering a proper dose of pungent liquid directly to the back of the throat without exposing the taste buds, soft palate or olfactory system so as to avoid the gag reflex, this immediately followed by a chaser dose (of, e.g., water or other pleasant tasting liquid) to wash it down.

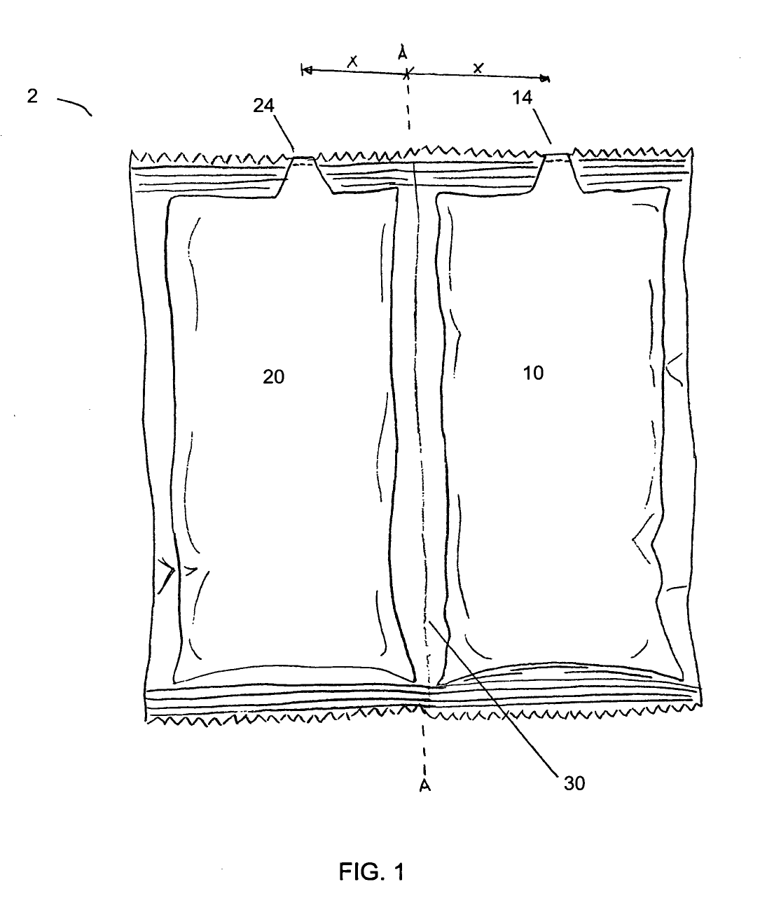

FIG. 1 is a diagram of one embodiment of the present invention which generally comprises a dual pouch dispenser formed between two sheets of plastic film, foil or similar flexible medium. For example, Low density Polyethylene (LDPE) is a well-suited film material that is tough, flexible and relatively transparent, and may be heat-sealed into the dual pouch dispenser configuration. Mylar™ or Cellophane™ will also suffice. It may also be possible to construct an effective applicator from a semi-rigid material such as laminated cardboard or the like.

A first chamber 10 is prefilled with an appropriate dosage of a first pungent fluid specifically chosen to excite the esophageal sphincter. Pungent is herein defined as that which is capable of contracting the esophageal sphincter at the back of the throat by imparting a sharp acrid sensation, and yet which can be safely digested. This can be achieved with a slightly acidic liquid that is generally recognized as safe (GRAS), and a most suitable expedient is vinegar. The pH of vinegar is typically in the range of 2.5 to 3, depending on the concentration of acetic acid, and the applicator embodiments described herein will employ cider vinegar. However, one skilled in the art should understand that other pungent liquids may be equally effective. The first chamber 10 is prefilled with an appropriate dosage of apple cider vinegar to be dispensed, and is sealed by fusing the plastic film together to seal the liquid between the films. A preferred dose is 3 ml.

An aperture 14 is formed in pouch 10 at its periphery. Aperture 14 is sealed at the time of manufacture by a barrier that seals the vinegar in pouch 10 but is burstable when sufficient pressure is applied to the pouch. The burst pressure of aperture 14 is substantially less than that of the remainder of the sealed periphery of pouch 10 to ensure that vinegar in pouch 10 is dispensed via the aperture.

A second pouch, 20 is affixed to first pouch 10 along at least one peripheral edge 30. Likewise, an aperture 24 is formed in pouch 20 at its periphery. Aperture 24 is sealed at the time of manufacture by a barrier that seals the chaser dose (e.g., water or other pleasant tasting liquid) in pouch 20 but is burstable when sufficient pressure is applied to the pouch. Preferably, the burst pressure of aperture 24 is slightly more than that of aperture 14 in pouch 10, but substantially less than that of the remainder of the sealed periphery of pouches 10 or 20 to ensure that the chaser dose in pouch 20 is dispensed via the aperture 24. The burst pressure of aperture 24 can be made more than that of pouch 10 by increasing the extent or integrity of the heat-seal barrier at the time of manufacture, and this is specifically done to ensure that when a user compresses the two pouches 10, 20 together, the vinegar pouch 10 will rupture first and empty its contents prior to the rupture of chaser pouch 20. This sequence of delivering one dose to the back of the user's throat followed by the other dose is an essential aspect of the present invention, and it is preferred to deliver the pungent liquid (vinegar) first followed by the chaser to the user's mouth, although the applicator is equally well-suited for the opposite sequence.

The liquids in the adjacent vessels 10, 20 are separately contained until the dual pouch dispenser 2 is raised to the user's mouth. It is intended that the dual pouch dispenser 2 be pointed toward the back of the user's throat, and then squeezed. The increased burst pressure of aperture 24 is more than that of aperture 14 and so the vinegar pouch 10 will rupture first and empty its contents just prior to the rupture of chaser pouch 20. Apple cider vinegar contained in the first pouch 10 will be applied directly to the back of the user's throat. As the user keeps squeezing the dual pouch dispenser 2, the second pouch 20 will empty and the chaser dose will be applied directly to the back of the user's throat, essentially washing the vinegar down. Thus the present invention effectively delivers a measured quantity of apple cider vinegar to the back of the mouth, and immediately flushes the vinegar down with a following quantity of water or other chaser liquid. The benefit of this approach is that the vinegar hits the esophagus less diluted, and is therefore potentially more effective. Further, the vinegar is delivered to the back of the throat (by virtue of the long narrow shape of the dual pouch dispenser 2 and the directional aperture 24) substantially beyond the taste buds or olfactory passages, and is immediately washed down before it becomes distributed in the mouth. Experience has shown that instead of 30 cc of diluted vinegar, 3 ml of undiluted vinegar delivered in the manner described above has the desired effect of temporarily relieving symptoms associated with heartburn. Similarly, it has been found that a 6 ml chaser of water avoids a gag reflex.

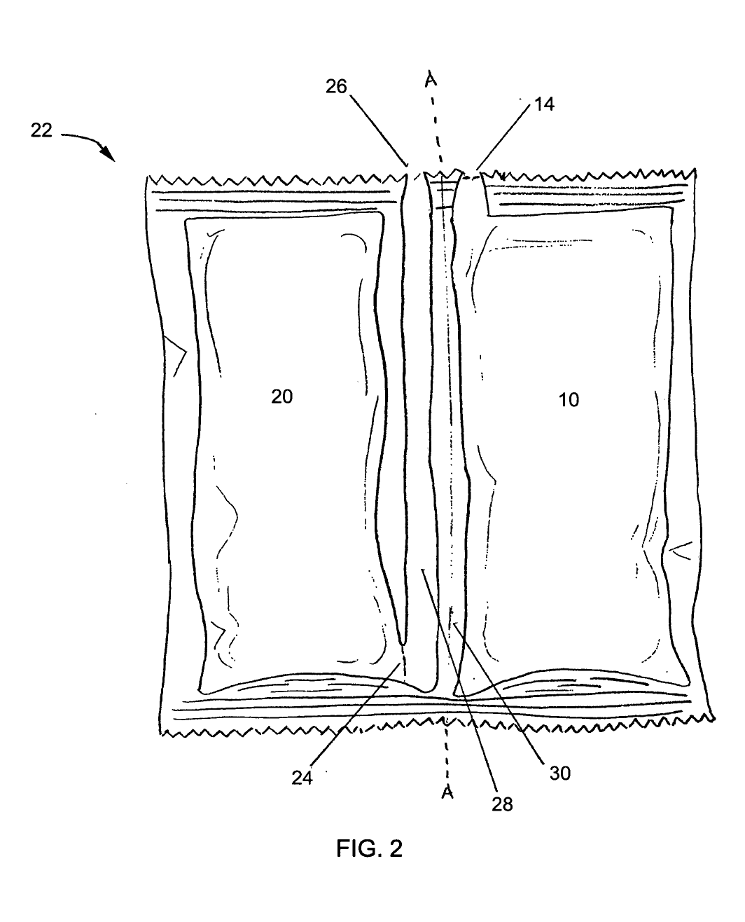

FIG. 2 is a front view of an alternative dual-pouch dispenser 12 similar to that of FIG. 1, but wherein the delay sequence is offset by a differential length in the flow paths rather than differential burst pressures. Specifically, the aperture 24 formed in pouch 20 is sealed at the time of manufacture by a barrier that seals the chaser dose in pouch 20 but is burstable when sufficient pressure is applied to the pouch. However, this aperture 24 empties at the bottom of the pouch into an elongate conduit 28 that runs upward to the top of the pouch 20. The aperture 14 formed in pouch 10 empties directly at the top of the pouch 10, where it is sealed at the time of manufacture by a barrier that seals the apple cider vinegar in pouch 10 but is burstable when sufficient pressure is applied to the pouch. The burst pressure of aperture 14 may be the same as that of aperture 24, and the pouches may rupture simultaneously when the dispenser is squeezed. However, the vinegar in pouch 10 will empty its contents first directly through aperture 14, while the contents of chaser pouch 20 will first need to wind its way through conduit 28 before emptying at opening 26, again accomplishing a delayed sequence of delivering first vinegar then chaser to the user's mouth.

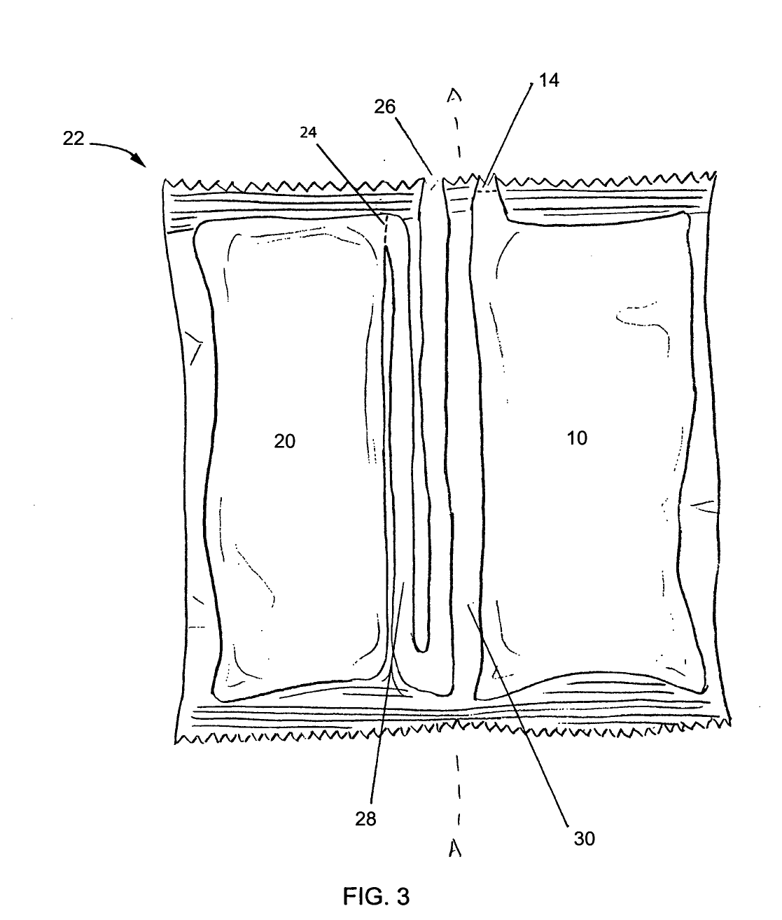

FIG. 3 is a front view of yet another alternative dual-pouch dispenser 22 similar to that of FIG. 2, but wherein the delay sequence is further offset by increasing the differential length in the flow paths. Aperture 24 is sealed at the time of manufacture by a barrier (dotted line) that seals the chaser in pouch 20 but is burstable when sufficient pressure is applied to the pouch. Aperture 24 empties at the top of the pouch but elbows down to the bottom then back up around to the top, in a double-length elongate conduit 28 emptying at the top of the pouch 20. Again, the aperture 14 formed in pouch 10 empties directly at the top of the pouch 10, where it is sealed at the time of manufacture by a barrier that seals the apple cider vinegar in pouch 10 but is burstable when sufficient pressure is applied to the pouch. The burst pressure of aperture 14 may be the same as that of conduit 28, and the pouches may rupture simultaneously when the dispenser is squeezed. However, the vinegar pouch 10 will empty its contents first directly through aperture 14, while the content of chaser pouch 20 must wind its way through conduit 28, again accomplishing a further-delayed sequence of delivering first vinegar then chaser to the user's mouth.

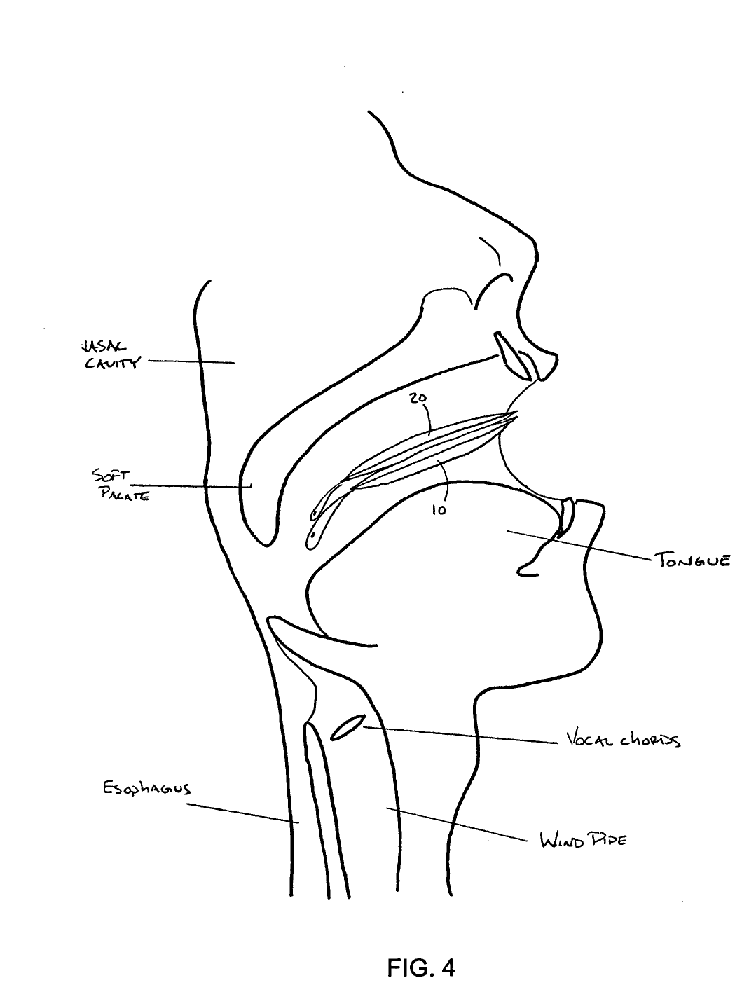

FIG. 4 is a diagram of the human mouth and throat illustrating the method of using the present invention for palatable prevention of acid reflux by delivering a throat rinse comprising the steps of providing a dual-compartment container formed with two discrete compartments, and a sequential delivery mechanism for first delivering a pre-measured dose of vinegar from one compartment directly to the back of the throat, and then a pre-measured dose of chaser from one compartment directly to the back of the throat to chase the vinegar. Using the embodiments described above, the dual-compartment container further comprises a first flexible chamber containing vinegar and having a first sealed aperture opening to the perimeter and a second flexible chamber containing chaser and having a second sealed aperture opening to the perimeter. The method further includes the application of pressure to burst the sealed apertures, either in succession to sequentially to first deliver the vinegar followed by the water chaser, or simultaneously to sequentially deliver first the vinegar directly through the aperture followed by the water chaser through a longer conduit. In either case the vinegar and chaser are discharged to the back of the throat directly and sequentially, the vinegar stimulating the lower esophageal sphincter (LES) to prevent or reduce acid reflux or GERD without exposing the taste buds, soft palate or olfactory system, immediately followed by a chaser of water or other pleasant tasting chaser to wash it down.

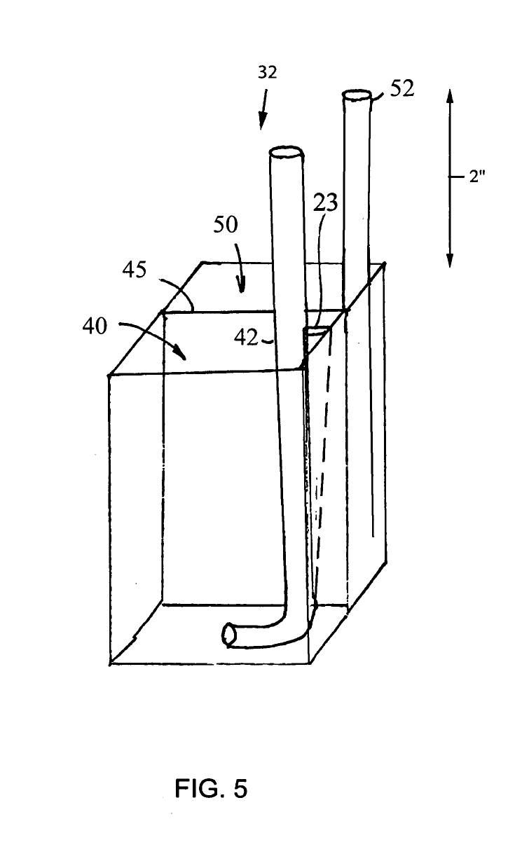

FIG. 5 is a perspective view of yet another alternative dispenser 32. The applicator 32 generally comprises a plastic dual-chamber vessel, which may be blow-molded thermoplastic or otherwise formed.

The vessel includes two side-by-side receptacles 40, 50 which may take any of a variety of shapes. The receptacles 40, 50 may be pre-filled with the appropriate dosages (chaser and vinegar) and fully sealed, or they may be open-topped for refilling by the user. In either case, the receptacles 40, 50 are bounded along at least five sides and are separated by a partition 45.

An integrally-molded first straw 42 enters one receptacle 40 and extends to the bottom wall, where its distal end lies along the bottom of the first receptacle 40 and remains open. An integrally-molded second straw 52 enters the other receptacle 50 and extends to the bottom wall, where its distal end lies along the bottom of the second receptacle 50 and remains open. The second straw 52 is aligned with the sidewalls of the second receptacle 50 (substantially vertical). The first straw 42, on the other hand, is angled forward at a positive offset in advance of the wall at an angle of approximately 10-15 degrees. While this implements one set of relative inclines, others are possible so long as the vinegar is poured out before the chaser dose is poured out, preferably with the chaser dose following immediately so as to wash the vinegar out of the mouth without delay.

To accomplished a rigid offset the first straw 42 may be supported by an angled strut 23, which may be integrally molded between the straw 42 and sidewall of vessel 40. The first vessel 40 is filled with a measured dose of apple cider vinegar. The second vessel 50 is filled with a chaser of water or any other pleasant-tasting liquid. The two straws 42, 52 are elongated and protrude approximately two inches past the tops of the vessels 40, 50. Initially the straws 42, 52 are tamper-sealed by removable caps or cellophane seals. The seals must be broken to use the device 32.

The liquids in the adjacent vessels 40, 50 are separately contained until the two-compartment vessel is tilted to the user's mouth. As the device 32 is rotated clockwise (from the position depicted in FIG. 5) the apple cider vinegar contained in the second vessel 50 will flow from its respective vessel 50 first out through the straw 52. This is because the other straw 42 takes longer to reach horizontal and so the chaser remains in the first vessel 40 while the other vessel 50 drains. However, as the user keeps tilting the second vessel 50 will empty and the chaser will begin to pour out of the first vessel 40, essentially washing the vinegar down the throat. Thus the present invention effectively delivers a measured quantity of apple cider vinegar to the back of the mouth, and immediately flushes the vinegar down with a following quantity of water or other chaser liquid. The benefit of this approach is that the vinegar hits the esophagus less diluted, and is therefore potentially more effective. Further, the vinegar is delivered to the back of the throat (by virtue of the elongate straws 42, 52) substantially beyond the taste buds or olfactory passages, and is immediately washed down before it becomes distributed in the mouth. Experience has shown that instead of 30 cc of diluted vinegar, 5 cc to 10 cc of undiluted vinegar delivered in the manner described above has the desired effect of temporarily relieving symptoms associated with heartburn.

If the present device is to be user-fillable (not pre-packaged) then the sidewalls of both vessels 40, 50 are preferable demarcated with measuring lines so that the user can fill accordingly with the prescribed doses, and the device will dispense only the measured amount when poured, especially accurate if the package as shown is be rotated clockwise to pour.

One skilled in the art will readily understand that variations in the device packaging are possible. For example, rather than a solid plastic container of glass or metal if desired. In addition, the device may be adapted to dispense small amounts of chaser (water or otherwise) both before and after the vinegar, only before the vinegar, or at the same time as vinegar. A burst of chaser before (in addition to after) seems to open the throat and may increase effectiveness. Moreover, the device may dispense diluted vinegar if so desired, with or without chaser.

Still other embodiments may include pumps as well as gravity fed measuring and dispensing chambers, as long as the vinegar is dispensed first to the back of the mouth followed by chaser to wash the vinegar out of the mouth as soon as the vinegar is dispensed.

In all such cases, the primary advantages of the above-described configuration are that the packaging is simple and inexpensive to produce, it need not have any moving parts, and the two vessels are independent so that each can deliver a different quantity of liquid.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

I claim:

1. A throat rinse delivery system, comprising:
a first chamber formed by two flexible sheets of material sealed together about the perimeter of said first chamber, and including a first sealed aperture along said perimeter which will burst under application of manual pressure to said first chamber;
a pre-measured dose of pungent liquid contained in said first chamber;
a second chamber formed by two flexible sheets of material sealed together about the perimeter of said second chamber, and including a second sealed aperture along said perimeter which will burst under application of manual pressure to said second chamber;
a pre-measured dose of chaser fluid contained in said second chamber;
whereby said second chamber is joined to said first chamber along at least one peripheral edge.

2. The throat rinse delivery system of claim 1 wherein said second sealed aperture is capable of withstanding greater applied pressure than said first sealed aperture such that said first sealed aperture will burst before said second sealed aperture thereby delivering said pungent liquid and chaser in a temporal sequence.

3. The throat rinse delivery system of claim 1 wherein said first sealed aperture is capable of withstanding greater applied pressure than said second sealed aperture such that said second sealed aperture will burst before said first sealed aperture thereby delivering said chaser and pungent liquid in a temporal sequence.

4. The throat rinse delivery system of claim 2 wherein said first chamber and second chamber are adjoining along a peripheral edge, and said first sealed aperture and said second sealed aperture are proximately located equidistance from said peripheral edge for alignment when said flexible material is folded about said joined edge.

5. The throat rinse delivery system of claim 2 wherein said pungent liquid is vinegar.

6. The throat rinse delivery system of claim 5 wherein said pungent liquid consists of undiluted apple cider vinegar.

7. A throat rinse delivery system, comprising:
a first chamber formed by two flexible sheets of material sealed together about the perimeter of said first chamber, and including a first sealed aperture along said perimeter constructed to burst under application of manual pressure to said first chamber;
a pre-measured dose of pungent liquid contained in said first chamber;
a second chamber formed by two flexible sheets of material sealed together about the perimeter of said second chamber, and including a second sealed aperture constructed to burst under application of manual pressure to said second chamber, said second sealed aperture leading to an elongate conduit opening along said perimeter;
a pre-measured dose of chaser fluid contained in said second chamber;
whereby said second chamber is joined to said first chamber along at least one peripheral edge.

8. The throat rinse delivery system of claim 7 wherein said premeasured dose of chaser fluid takes longer to discharge through said elongate conduit to said perimeter when said first and second sealed apertures burst under pressure.

9. The throat rinse delivery system of claim 7 wherein said sheets are selectively sealed together to form said conduit in fluid communication with said second chamber via said second aperture.

10. The throat rinse delivery system of claim 7 wherein said pungent liquid is vinegar.

11. The throat rinse delivery system of claim 10 wherein said pungent liquid consists of apple cider vinegar.

12. The throat rinse delivery system of claim 11 wherein said vinegar is provided in said first chamber in a pre-measured 3 ml dose.

13. A method of delivering a throat rinse comprising the steps of:
providing a dual-compartment container formed with two discrete compartments;
providing a pre-measured dose of pungent liquid in one of said two compartments;
providing a pre-measured dose of chaser liquid in another of said two compartments;
sequentially delivering said pre-measured dose of pungent liquid from said one compartment directly to the back of the throat, and then the pre-measured dose of chaser liquid from said other compartment directly to the back of the throat to chase the pungent liquid.

14. The method of delivering a throat rinse according to claim 13, wherein said dual-compartment container is formed of flexible sheet material in two discrete compartments each having a sealed aperture, and said sequential delivery step further comprises applying manual pressure to said first and second chambers to burst the sealed apertures and release said pungent liquid and said chaser.

15. The method of delivering a throat rinse on claim 13 wherein the seal of said second aperture is capable of withstanding greater applied pressure than the seal of said first aperture.

16. The method of delivering a throat rinse on claim 13 wherein said pungent liquid is apple cider vinegar.

17. A method of delivering a throat rinse comprising the steps of:
providing a first chamber having a first sealed aperture constructed to burst under application of manual pressure, said first chamber containing a first fluid material;
providing a second chamber integral to said first chamber and having a second sealed aperture constructed to burst under application of manual pressure to said second chamber, said second chamber containing a second fluid material;
providing a pre-measured dose of pungent liquid in said first chamber;
providing a pre-measured dose of chaser liquid in said second chamber;
inserting said first and second chambers into a mouth such that said apertures are directed to the back of the throat;
applying pressure to said chambers whereby said seals of said first and second apertures burst and said first liquid material is discharged to said throat directly.

18. The method of delivering a throat rinse as in claim 17, wherein said dose of pungent liquid is dispensed prior to said dose of chaser liquid.

19. The method of delivering a throat rinse as in claim 18 wherein said first liquid is cider vinegar.

20. A device for reducing acid indigestion and reflux, comprising:
a drinking vessel defined by two side-by-side receptacles, including a first receptacle and a second receptacle;
a pair of fixedly attached straws each entering a corresponding receptacle, said straws being fixedly attached to the inner walls of said vessel, opening at a bottom of the respective receptacles, and extending side-by-side to adjacent distal tips at least one inch remote from said vessel, whereby one of said straws entering said second receptacle is oriented at a fixed angle greater than the other of said straws entering said first receptacle so that contents of said first receptacle pour first immediately followed by the contents of the second receptacle;
a dose of liquid undiluted apple cider vinegar resident in said first receptacle; and
a dose of liquid chaser resident in said second receptacle.
